(19) United States
(12) Patent Application Publication
(10) Pub. No.: US 2006/0188978 A1
(43) Pub. Date: Aug. 24, 2006

(54) Products and processes for waste control

(76) Inventor: Daniel T. Grant, Modesto, CA (US)

Correspondence Address: ALAN J. HOWARTH, P.O. BOX 1909, SANDY, UT, 84091-1909, US

(21) Appl. No.: 11/066,122

(22) Filed: Feb. 24, 2005

(51) Int. Cl. C12P 1/04; C12N 1/20; C12N 1/18

(52) U.S. Cl. 435/252.5; 435/170; 435/254.21

(57) ABSTRACT

Products and methods for controlling animal wastes are disclosed. The methods include making an inoculum containing a mixture of Bacillus species; mixing the inoculum with animal wastes and then incubating the mixture to result in an enriched culture; and then applying the enriched culture to the animal wastes. The inoculum for controlling wastes includes a mixture of Bacillus species in a liquid medium in which the mixture of Bacillus species were incubated.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

This invention relates to products and processes for waste control. More particularly, this invention relates to products and processes for controlling animal wastes in lagoons and other sites where animal wastes are stored.

Manure and other livestock waste has the potential to be either a valuable resource or a major health threat and pollutant, depending on its management. When not treated or improperly handled, manure nutrients, primarily nitrogen, are potential pollutants of air and water. Thus, manure management in the livestock industry has recently received a great deal of attention. Focus on this issue has occurred for several reasons. The major reason is recent advancements in science have clarified the connection between manure and health and pollution. In some situations, declining water quality has been directly linked to poor manure management.

Ground water contamination resulting from nitrate leaching and runoff water poses a health risk when it is consumed by humans or animals. Health problems associated with nitrate result primarily after the nitrates enter the body and are converted by bacteria to nitrites. High nitrite levels may cause a condition known as methemoglobinemia, also known as “blue baby syndrome.” Methemoglobinemia occurs when nitrites, or other oxidants, oxidize the iron in hemoglobin from its normal ferrous (Fe²⁺) form to the ferric (Fe³⁺) form, thus converting hemoglobin to methemoglobin, a brown pigment. Since methemoglobin is incapable of binding molecular oxygen, the ultimate result of methemoglobinemia is suffocation. Therefore, high levels of nitrogen in areas in which ground water makes up a large portion of the drinking water are a real and valid danger.

Further, additions of raw or semi-treated manure to a lake or stream will significantly degrade water quality. Manure additions increase the nitrogen and organic matter content of the lakes and streams, contributing to excessive algal and aquatic plant growth. This growth has a high oxygen demand, resulting in gradual depletion of the water's oxygen supply. This algal and plant bloom adversely affects fish and other aquatic life and has a negative impact on the beneficial use of water resources for drinking or recreation. If oxygen concentrations fall below a critical level, fish and other aquatic species die in massive numbers.

Not only are there human health concerns and danger to area aquatic species, but odor also presents a major problem in raising livestock. Odor issues often create tension between livestock producers and neighbors or entire communities. Because odors are produced during the anaerobic decomposition of manure, the surest way to decrease odors is to provide oxygen to the manure via aeration. However, aeration is not practical, is very expensive, and requires large amounts of energy. Thus, many livestock producers do not adequately treat manure for reducing odors.

Focus on manure management is the result of the public's increasing environmental concerns. For a majority of the public, the words “manure” and “pollution” have become synonymous. Due to the actual and perceived problems with manure, the public is demanding livestock producers be accountable for manure handling and treatment. In fact, an increasing number of federal lawsuits have been filed against livestock producers claiming that the producers have violated the U.S. Clean Water Act, Clean Air Act, and National Pollution Discharge Elimination System Permit Programs. Therefore, manure management and treatment is becoming a major factor in local, national, and international livestock production. It is clear that livestock producers must effectively treat their manure and other waste products to avoid the aforementioned problems and possibly even expensive and lengthy lawsuits.

There are many treatment systems currently used to handle the massive amounts of manure generated by livestock, including liquid-solid separation, composting, oxygen ditches, physical removal, digging deeper lagoons, and the like. However, there are many problems and drawbacks associated with each and, on the whole, treatment systems are not typically cost effective.

Liquid-solid separation systems, such as settling basins and mechanical separators, provide a means of separating nutrients from the waste stream. However, the separation of nutrients requires relatively large mechanical separators, which can be quite costly and require substantial amounts of energy.

Composting reduces and stabilizes the organic matter, but can require quite a large amount of time to properly compost the manure into a uniform, dry, and odorless soil conditioner.

Oxidation ditches stabilize and reduce the organic matter in the waste material. These systems, however, are relatively uncommon and typically uneconomical due to their high energy demands.

Some livestock producers have resorted to building miles and miles of pipe line to transport waste material to land some distance from the farm itself. However, this is a cumbersome method of handling manure, as well as an expensive one. Further, neighboring communities are threatened by pipe bursts or leaks.

Other producers have resorted to digging deeper lagoons, as much as 20-25 feet deep, as opposed to the standard 8-10 feet deep lagoon, to store waste material. However, the increase in depth dramatically decreases the surface-to-volume ratio, resulting in exceedingly high nitrogen levels.

Therefore, prior art products and methods for treating animal wastes are known and are generally suitable for their limited purposes, but they possess certain inherent deficiencies that detract from their overall utility in safeguarding health and the environment and in reducing noxious odors.

In view of the foregoing, it will be appreciated that providing products and methods for treating animals wastes that address these deficiencies would be a significant advancement in the art.

### BRIEF SUMMARY OF THE INVENTION

An illustrative method for controlling animal wastes according to the present invention comprises:

(a) preparing an inoculum comprising a mixture of Bacillus species and having a selected volume and a cell density of at least about 10⁵ cells/ml;

(b) mixing the selected volume of the inoculum with about 0.1 to 10 times the selected volume of the animal wastes to be controlled and incubating the resulting mixture at ambient temperatures for about 12 hours to 14 days to result in an enriched culture; and

(c) applying the enriched culture to the animal wastes to be controlled.

Illustratively, the Bacillus species are selected from the group consisting of Bacillus subtilis, Bacillus megaterium, Bacillus polymyxa, Bacillus licheniformis, Bacillus uniflagellatus, Bacillus lacterosporus, Bacillus chitinosporus, Bacillus amyloliquefaciens, Bacillus pumilus, Bacillus stearothermophilus, Bacillus mycoides, Bacillus sphaericus, Bacillus coagulans, Bacillus thuringiensis, Bacillus lentus, Bacillus circulars, Bacillus badius, and Bacillus firmus.

The inoculum typically has a cell density of at least about 10⁸ cells/ml, and, more typically, at least about 10¹² cells/ml. The inoculum is prepared by culturing a composition comprising about 0.01 to 10 parts by weight of the mixture of Bacillus species, about 1 to 20 parts by weight of complex microbiological culture medium, and about 70 to 98.99 parts by weight of water. The complex microbiological culture medium illustratively comprises whey protein concentrate, fish protein, poultry meal, molasses, or mixtures thereof, but can also comprise humic acid and/or Saccharomyces cerevisiae.

An additional illustrative embodiment of the invention according to the present invention comprises an enriched bacterial culture for use in controlling animal wastes, the enriched bacterial culture comprising one volume of a mixture of Bacillus species having a cell density of at least about 10⁵ cells/ml in a liquid medium comprising about 0.1 to 10 volumes of a sample of the animal wastes to be controlled in which the mixture of Bacillus species was cultured for about 12 hours to 14 days.

### DETAILED DESCRIPTION

Before the present products and methods for treating animal wastes are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to “an inoculum” includes reference to two or more of such inocula, reference to “a culture medium” includes reference to two or more of such culture media, and reference to “a Bacillus species” includes reference to a mixture of two or more of such Bacillus species.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

Dairy lagoons illustrate the problems mentioned above for disposal of animal wastes. Dairy lagoons contain an extremely high organic nutrient holding capacity or cation exchange. These elements include potassium, calcium, magnesium and sulfur. Further, dairy lagoons contain high amounts of macronutrients, principally nitrogen and phosphates.

The products and methods of the present invention relate to mixtures of Bacillus strains that allow the elements and macronutrients to undergo biological exchange in dairy lagoons before the elements and macronutrients enter the fields and groundwater surrounding the lagoons. Therefore, the elements and macronutrients are readily available for plant absorption through root hairs.

Intrinsically, no two dairies are the same. The products and methods of the present invention obtain bacterial mixtures that are sensitized to a specific waste lagoon. The sensitized bacterial mixture is then released directly into the waste lagoon. The lagoon becomes biologically active, which results in churning solids from the lagoon floor. The bacterial mixture digests molds and fungi and, further, hinders insect reproduction. Moreover, ammonia and methane gases are substantially reduced. Benefits of these products and methods include increasing nutrient efficiency, decreasing solid waste disposal and lagoon dredging, eliminating offensive odors, diminishing environmental concerns, reducing fly and mosquito numbers, and cleaning and drying flush lanes, thus reducing injuries to cattle.

Bacillus species have been found to produce enzymes that catabolize proteins, complex carbohydrates, fats, oil, and organic acids and further that degrade decaying organic matter. Bacillus species have also been found to be excellent for industrial applications. Further, many Bacillus species are generally regarded as safe (GRAS) according to the regulations of the U.S. Food and Drug Administration. Bacillus species that can be used according to the present invention for controlling animal wastes include Bacillus subtilis, Bacillus megaterium, Bacillus polymyxa, Bacillus licheniformis, Bacillus uniflagellatus, Bacillus lacterosporus, Bacillus chitinosporus, Bacillus amyloliquefaciens, Bacillus pumilus, Bacillus stearothermophilus, Bacillus mycoides, Bacillus sphaericus, Bacillus coagulans, Bacillus thuringiensis, Bacillus lentus, Bacillus circulars, Bacillus badius, and Bacillus firmus.

A mixture of Bacillus species is obtained by mixing various Bacillus species in selected ratios and then culturing the mixture according to methods well known in the art. Alternatively, the Bacillus species can be cultured separately and then the cultured bacteria can be mixed in selected ratios. These methods include regulating the temperature; hydrogen ion concentration (pH); partial pressure of oxygen; mineral salt concentration and complex media ingredient concentration for regulating the supply of nitrogen, phosphorus, sulfur, calcium, iron, heavy metals, and the like; culture period; and stirring speed, according to methods well known in the art. These culture variables can be varied individually or in combination. Generally, the culture conditions are in the following ranges: temperature, 10-70° C.; pH, 1-10; oxygen concentration, 0-48 mg/l; culture period, 5 hours to 14 days; stirring speed, 0-1500 revolutions per minute (on a laboratory scale).

To obtain high cell densities, it is well known that complex media result in much higher cell densities than can be obtained by merely supplying minimal salts. These complex media typically contain protein and carbohydrate sources, which also contain sufficient salts to supply the nutritional needs of the microorganisms. Protein and carbohydrate sources that can be used according to the present invention include poultry meal, whey protein concentrate, fish protein, molasses, and the like, and mixtures thereof. In addition, it has been found that adding humic acid is beneficial to growth of the bacteria. Still further, it has been found that adding brewer's yeast (Saccharomyes cerevisiae) is also beneficial for making the composition that controls animal wastes.

Illustratively, the inoculum used for preparing the composition according to the present invention contains a mixture of Bacillus species having a cell density of at least about 10⁵ cells/ml, and typically at least about 10⁸ cells/ml, and more typically at least about 10¹² cells/ml. Typically, this inoculum is prepared by mixing selected Bacillus species, water, and microbiological culture media and then incubating the resulting mixture for a selected period of time at room temperature with agitation, such as by pumping air through the mixture, until the selected cell density is obtained. Cell density is determined according to methods well known in the art. Typically, the inoculum is prepared by mixing about 0.01 to 10 parts by weight of bacteria, about 1 to 20 parts by weight of complex microbiological media, and about 70 to 98.99 parts by weight of water.

The inoculum is then mixed with water and the microbiological culture media to result in a composition having about 10⁵ to 10⁸ cells/ml prior to additional incubation and culturing of the mixed Bacillus species. Next, about 0.1 to 10 volumes of animal waste, typically about 1 to 4 volumes, and more typically about 3 to 4 volumes, are added to the composition. For example, if the volume of mixed Bacillus species is about 1893 liters (i.e., about 500 gallons), then about 5678 liters (i.e., about 1500 gallons or 3 volumes) of the animal waste can be mixed with the bacterial composition. This composition is then incubated without agitation at ambient temperatures for a period of time, usually hours to days, to obtain an enriched culture, which is enriched for bacteria that are capable of degrading the animal wastes and reducing or eliminating the noxious odors by catabolizing the organic molecules that are responsible for such noxious odors.

This step of producing the enriched culture can be repeated, if desired. That is, a portion of the enriched culture can be mixed with fresh water and culture media, and then a selected amount of animal wastes is added and the resulting mixture is incubated again, similar to the procedure described above, to result in a twice-enriched or super-enriched culture. It will be appreciated by those skilled in the art that such enrichment process can again be repeated as often as desired.

Once the enriched culture has been prepared, it is ready to be applied to the animal wastes. For example, if the animal wastes are contained in a lagoon, the enriched culture (or super-enriched culture) can be sprayed onto the surface of the lagoon or otherwise mixed with the contents of the lagoon for obtaining good coverage of the lagoon. The amount of enriched culture that should be added to the lagoon is easily determined by a person of ordinary skill in the art without the need for undue experimentation. Addition of an appropriate amount of enriched culture will result in vigorous production of gas as the bacteria metabolize the wastes. Further, the bacteria will substantially control the odors associated with the lagoon within two weeks. Addition of an excess of enriched culture will result in solids from the floor of the lagoon being raised to the surface of the lagoon due to the large amount of gas being produced by the bacteria. This condition is sometimes termed being “upside down.” Remedies for an upside down lagoon include (1) doing nothing, wherein the production of gas by the bacteria will gradually diminish and permit the solids to sink to the lagoon floor again, and (2) adding even more enriched culture to the lagoon, wherein the solids will be further metabolized by the bacteria and will sink to the floor of the lagoon. The production of gas by the bacteria added to the lagoon results in mixing of the contents of the lagoon, which aids in mixing of the bacteria with the wastes.

Since wastes will generally continue to be added to the lagoon, it is convenient to periodically add fresh enriched culture to the lagoon. One illustrative embodiment of this principle involves placing two bacterial growth tanks near a lagoon to be treated. Two weeks before beginning treatment of the lagoon, bacterial inoculum, microbiological growth media, water, and an aliquot of liquid waste from the lagoon are placed in one of the tanks, according to the procedure described above. This composition is incubated at ambient temperatures for two weeks, resulting in production of a first enriched culture. Once the first enriched culture is ready for use, about one-fourteenth of the first enriched culture is applied to the lagoon each day for fourteen days. At the end of a two-week period, the first enriched culture will be depleted. On the first day that the first enriched culture is applied to the lagoon, the second tank is filled with inoculum, microbiological growth media, water, and an aliquot of waste from the lagoon. This second mixture is permitted to incubate at ambient temperatures for 14 days, to result in production of a second enriched culture in the same manner that the first enriched culture was produced. Once the first enriched culture is depleted, about one-fourteenth of the second enriched culture is added to the lagoon each day for fourteen days. While the second enriched culture is being used for treating the lagoon, the first tank is again used for preparation of a third enriched culture. This process is repeated indefinitely, that is, while one of the tanks and its enriched culture is being used to treat the lagoon, the other tank is used for preparation of a new enriched culture.

### EXAMPLES

The following examples are given to illustrate the invention, but are not to be construed as limitations on the scope of the invention. The scope of the invention is to be determined by the claims and equivalents thereof.

### Example 1

A composition is prepared by mixing 0.03 parts by weight of Bacillus subtilis, 0.18 parts by weight of poultry meal (spray dried), 0.18 parts by weight of whey protein concentrate, 0.23 parts by weight of brewer's yeast, 0.23 parts by weight of fish protein, 0.35 parts by weight of humic acid (derived from leonardite), 4.75 parts by weight of molasses, and 94.05 parts by weight of water. This mixture is incubated at about 32.2° C. (i.e., about 90° F.) for 7 days with agitation by pumping air through the mixture. The resulting culture is then ready to be subcultured.

Accordingly, about 1893 liters (500 gallons) of the mixture is diluted with 5678 liters (1500 gallons) of liquid lagoon waste from a manure lagoon located on a dairy. The resulting mixture is then cultured at ambient temperatures for about 14 days without agitation to result in an enriched culture. The enriched culture is then applied to the lagoon.

### Example 2

The procedure of Example 1 is repeated except that Bacillus megaterium is substituted for Bacillus subtilis.

### Example 3

The procedure of Example 1 is repeated except that Bacillus polymyxa is substituted for Bacillus subtilis.

### Example 4

The procedure of Example 1 is repeated except that Bacillus licheniformis is substituted for Bacillus subtilis.

### Example 5

The procedure of Example 1 is repeated except that Bacillus uniflagellatus is substituted for Bacillus subtilis.

### Example 6

The procedure of Example 1 is repeated except that Bacillus lactosporus is substituted for Bacillus subtilis.

### Example 7

The procedure of Example 1 is repeated except that Bacillus chitinosporus is substituted for Bacillus subtilis.

### Example 8

The procedure of Example 1 is repeated except that a mixture of Bacillus subtilis and Bacillus megaterium is substituted for Bacillus subtilis.

### Example 9

The procedure of Example 1 is repeated except that a mixture of Bacillus subtilis and Bacillus polymyxa is substituted for Bacillus subtilis.

### Example 10

The procedure of Example 1 is repeated except that a mixture of Bacillus subtilis and Bacillus licheniformis is substituted for Bacillus subtilis.

### Example 11

The procedure of Example 1 is repeated except that a mixture of Bacillus subtilis and Bacillus uniflagellatus is substituted for Bacillus subtilis.

### Example 12

The procedure of Example 1 is repeated except that a mixture of Bacillus subtilis and Bacillus lacterosporus is substituted for Bacillus subtilis.

### Example 13

The procedure of Example 1 is repeated except that a mixture of Bacillus subtilis and Bacillus chitinosporus is substituted for Bacillus subtilis.

### Example 14

The procedure of Example 1 is repeated except that a mixture of Bacillus subtilis, Bacillus megaterium, and Bacillus polymyxa is substituted for Bacillus subtilis.

### Example 15

The procedure of Example 1 is repeated except that a mixture of Bacillus subtilis, Bacillus megaterium, Bacillus polymyxa, and Bacillus licheniformis is substituted for Bacillus subtilis.

### Example 16

The procedure of Example 1 is repeated except that a mixture of Bacillus subtilis, Bacillus megaterium, Bacillus polymyxa, Bacillus licheniformis, and Bacillus uniflagellatus is substituted for Bacillus subtilis.

### Example 17

The procedure of Example 1 is repeated except that a mixture of Bacillus subtilis, Bacillus megaterium, Bacillus polymyxa, Bacillus licheniformis, Bacillus uniflagellatus, and Bacillus lacterosporus is substituted for Bacillus subtilis.

### Example 18

The procedure of Example 1 is repeated except that a mixture of Bacillus subtilis, Bacillus megaterium, Bacillus polymyxa, Bacillus licheniformis, Bacillus uniflagellatus, Bacillus lacterosporus, and Bacillus chitinosporus was substituted for Bacillus subtilis. The enriched culture was applied to a dairy lagoon daily for 14 days.

The subject matter claimed is:

1. A method for controlling animal wastes comprising:
(a) preparing an inoculum comprising a mixture of Bacillus species and having a selected volume and a cell density of at least about 10⁵ cells/ml;
(b) mixing the selected volume of the inoculum with about 0.1 to 10 times the selected volume of the animal wastes to be controlled and incubating the resulting mixture at ambient temperatures for about 12 hours to 14 days to result in an enriched culture; and
(c) applying the enriched culture to the animal wastes to be controlled.

2. The method of claim 1 wherein the Bacillus species are selected from the group consisting of Bacillus subtilis, Bacillus megaterium, Bacillus polymyxa, Bacillus licheniformis, Bacillus uniflagellatus, Bacillus lacterosporus, Bacillus chitinosporus, Bacillus amyloliquefaciens, Bacillus pumilus, Bacillus stearothermophilus, Bacillus mycoides, Bacillus sphaericus, Bacillus coagulans, Bacillus thuringiensis, Bacillus lentus, Bacillus circulars, Bacillus badius, and Bacillus firmus.

3. The method of claim 1 wherein the cell density of the inoculum is at least about 10⁸ cells/ml.

4. The method of claim 1 wherein the cell density of the inoculum is at least about 10¹² cells/ml.

5. The method of claim 1 wherein preparing the inoculum comprises culturing a composition comprising about 0.01 to 10 parts by weight of the mixture of Bacillus species, about 1 to 20 parts by weight of complex microbiological culture medium, and about 70 to 98.99 parts by weight of water.

6. The method of claim 5 wherein the complex microbiological culture medium comprises whey protein concentrate, fish protein, poultry meal, molasses, or mixtures thereof.

7. The method of claim 6 wherein the complex microbiological culture medium further comprise humic acid.

8. The method of claim 7 wherein the complex microbiological culture medium further comprises Saccharomyces cerevisiae.

9. The method of claim 6 wherein the microbiological culture media further comprises Saccharomyces cerevisiae.

10. The method of claim 1 further comprising subculturing the enriched culture by taking an aliquot of the enriched culture; adding water, complex microbiological culture medium, and a selected amount of animal wastes; and then incubating the resulting mixture to result in a super-enriched culture.

11. The method of claim 10 further comprising subculturing the super-enriched culture by taking an aliquot of the super-enriched culture; adding water, microbiological culture media, and a selected amount of animal wastes; and then incubating the resulting mixture.

12. The method of claim 1 wherein the mixture of the composition and the animal wastes is incubated for about 12 hours to 14 days.

13. The method of claim 1 wherein applying the enriched culture comprises spraying the enriched culture on a waste-containing lagoon.

14. The method of claim 1 wherein applying the enriched culture comprises mixing the enriched culture with a waste-containing lagoon.

15. The method of claim 1 wherein the selected volume of the inoculum is mixed with about 1 to 4 times the selected volume of the animal wastes to be controlled.

16. The method of claim 15 wherein the selected volume of the inoculum is mixed with about 3 to 4 times the selected volume of the animal wastes to be controlled.

17. The method of claim 1 further comprising applying the enriched culture to the animal wastes to be controlled on a daily basis.

18. An enriched bacterial culture for use in controlling animal wastes, the enriched bacterial culture comprising one volume of a mixture of Bacillus species having a cell density of at least about 10⁵ cells/ml in a liquid medium comprising about 0.1 to 10 volumes of a sample of the animal wastes to be controlled in which the mixture of Bacillus species was cultured for about 12 hours to 14 days.

19. The enriched bacterial culture of claim 18 wherein the liquid medium comprises about 1 to 4 volumes of the sample of the animal wastes to be controlled.

20. The enriched bacterial culture of claim 19 wherein the liquid medium comprises about 3 to 4 volumes of the sample of the animal wastes to be controlled.
